(19) United States
(12) Patent Application Publication
(10) Pub. No.: US 2026/0193583 A1
(43) Pub. Date: Jul. 9, 2026

(54) CELL CULTURE VESSEL INCLUDING A TWO-DIMENSIONAL NEGLIGIBLE VOLUME SPARGER

(71) Applicant: Corning Incorporated, Corning, NY (US)

(72) Inventors: Christopher Bowman Horner, Santa Cruz, CA (US); Gregory Roger Martin, Acton, ME (US)

(21) Appl. No.: 19/134,541

(22) PCT Filed: Nov. 17, 2023
(86) PCT No.: PCT/US2023/080219

§ 371 Date: May 30, 2025

Related U.S. Application Data
(60) Provisional application No. 63/428,815, filed on Nov. 30, 2022.

(51) Int. Cl. C12M 1/00

(52) U.S. Cl. CPC C12M 29/06; C12M 23/26

(57) ABSTRACT

An exemplary cell culture vessel is described including a housing defining a cavity operable to maintain a cell culture and an open end fluidly connected to the cavity, the open end surrounded by a peripheral edge of the housing, and a sparger coupled to the open end at the peripheral edge. The sparger includes a film having a first peripheral edge portion and a plate having a second peripheral edge portion. The first peripheral edge portion sealed to the second peripheral edge portion such that the film is spaced apart from the plate by less than about 100 micrometers to form a fluid passageway between the film and the plate. The film further includes a plurality of spaced apart holes. A method of using the cell culture vessel is also described.

### CROSS-REFERENCE TO RELATED APPLICATIONS

This application claims the benefit of priority under 35 U.S.C. § 119 of U.S. Provisional Application Ser. No. 63/428815 filed on Nov. 30, 2022, the content of which is relied upon and incorporated herein by reference in its entirety.

### FIELD

The present disclosure relates to a method and a cell culture vessel. The present disclosure relates more specifically to a cell culture vessel with a sparger.

### BACKGROUND

One common method used for supplying a gas (e.g., air, oxygen) to cell culture vessels is the use of a sparger. Bubbles produced in a liquid medium in the vessel from a sparger help to agitate and oxygenate the liquid medium (and cells therein) in the cell culture vessel. Typically, spargers are pipes extending into a volume of the vessel, the pipes configured with orifices from which the gas escapes the pipe. However, the relatively large cross-sectional area of these pipes and/or associated manifolding may allow backflow or retention of the liquid medium in the pipes. Additionally, such large, three-dimensional structures may require complex fabrication steps, excessive amounts of material for fabrication, and may provide limited flexibility, therefore being prone to damage during shipping and/or use in flexible vessels like bags.

### SUMMARY

An exemplary embodiment of the present disclosure provides a cell culture vessel. The cell culture vessel may include a housing defining a cavity operable to maintain a cell culture and an open end fluidly connected to the cavity, the open end surrounded by a peripheral edge of the housing. The cell culture vessel may include a sparger coupled to the open end at the peripheral edge, the sparger including a film including a first peripheral edge portion and a plate including a second peripheral edge portion, the first peripheral edge portion sealed to the second peripheral edge portion such that the film is spaced apart from the plate by less than about 100 micrometers to form a fluid passageway between the film and the plate, the film further including a plurality of spaced apart holes extending through a thickness of the film, the fluid passageway in fluid communication with the cavity through the plurality of holes.

In some embodiments, the thickness of the film may be less than about 2.5 millimeters. In some embodiments, the film may include a polymer. In some embodiments, a diameter of each hole of the plurality of holes may be less than about 1 millimeter. In some embodiments, the cell culture vessel may further include a gas line in fluid communication with the fluid passageway. In some embodiments, the cell culture vessel may further include a one-way valve operable to allow a flow of fluid into the fluid passageway in a first direction and prevent a flow of fluid from the fluid passageway in a second direction. In some embodiments, the one-way valve is positioned in the gas line. In some embodiments, the gas line is fixedly attached between the film and the plate. In some embodiments, the film may comprise a first major surface facing the cavity, and the first major surface may be non-planar. In some embodiments, the first major surface may be a treated surface configured to at least one of enhance or inhibit cell attachment to the first major surface. In some embodiments, the treated first major surface may comprise a net negative surface charge. In some embodiments, the treated first major surface may comprise a coating. In some embodiments, a wall of the housing may be flexible. In some embodiments, the film and the plate may be sealed to each other by at least one of an airtight adhesive, heat sealing, heat staking, ultrasonic welding, or solvent bonding.

An exemplary embodiment of the present disclosure provides a method of sparging a cell culture. The method may include depositing the cell culture into a cell culture vessel, the cell culture vessel including a housing defining a cavity operable to maintain the cell culture and an open end fluidly connected to the cavity, the open end surrounded by a peripheral edge of the housing, and a sparger coupled to the open end at the peripheral edge, the sparger including a film including a first peripheral edge portion and a plate including a second peripheral edge portion sealed to the first peripheral edge portion such that the film is spaced apart from the plate by less than about 100 μm to form a fluid passageway between the film and the plate, the film further including a plurality of spaced apart holes extending through a thickness of the film, the fluid passageway in fluid communication with the cavity through the plurality of holes. The method may further include flowing a gas into the fluid passageway, the gas issuing from the plurality of holes into the housing.

In some embodiments, a diameter of each hole of the plurality of holes may be less than about 1000 micrometers. In some embodiments, the gas may flow through a gas line extending between the fluid passageway and a gas source. In some embodiments, the gas may flow through a one-way valve before entering the fluid passageway. In some embodiments, the gas may be a first gas, the method further including flowing a second gas different from the first gas into the cavity. In some embodiments, the film may comprise a first major surface facing the cavity, the first major surface treated to at least one of enhance or inhibit cell attachment to the first major surface. In some embodiments, the depositing of the cell culture may comprise depositing an adherent cell culture and a suspension cell culture.

An exemplary embodiment of the present disclosure provides a method of forming a cell culture sparger. The method may include positioning a film including a first peripheral edge portion and a plurality of holes extending through a thickness of the film opposite a plate including a second peripheral edge portion and sealing the first peripheral edge portion to the second peripheral edge portion such that the film is spaced apart from the plate by a distance less than about 100 micrometers to form a fluid passageway between the film and the plate.

In some embodiments, the sealing may comprise at least one of an airtight adhesive, heat sealing, heat staking, ultrasonic welding, or solvent bonding. In some embodiments, the method may further include treating a surface of the film to at least one of enhance or inhibit cell attachment to the surface. In some embodiments, the treating may comprise a corona-gas treatment. In some embodiments, the treated surface may comprise a net negative charge after the treating. In some embodiments, the treating may comprise applying a coating to the surface. In some embodiments, the plurality of holes may be formed by laser ablation. In some embodiments, the film may comprise a first major surface facing the fluid passageway and a second major surface opposite the first major surface, the second major surface including one or more protrusions extending therefrom. In some embodiments, the film may comprise a first major surface facing the fluid passageway and a second major surface opposite the first major surface, and the second major surface may be non-planar.

The following will describe embodiments of the present disclosure. However, the present disclosure is not limited to the described embodiments and various modifications of the disclosure are possible without departing from the basic principles described herein.

### BRIEF DESCRIPTION OF THE DRAWING(S)

Various embodiments are disclosed, by way of example only, with reference to the accompanying schematic drawings below in which corresponding reference symbols indicate corresponding parts. Like drawing numbers on different drawing views identify identical, or functionally similar, structural elements.

FIG. 1 is a cross-sectional view of an exemplary cell vessel suitable for use in practicing exemplary embodiments of the present disclosure;

FIG. 2 is another cross-sectional view of an exemplary cell vessel suitable for use in practicing exemplary embodiments of the present disclosure;

FIGS. 3A-3F are top views of an exemplary film suitable for use in practicing exemplary embodiments of the present disclosure;

FIGS. 4A-4C are cross-sectional views of an exemplary film suitable for use in practicing exemplary embodiments of the present disclosure; and FIGS. 5A-5C are top views of elements of an exemplary film suitable for use in practicing exemplary embodiments of the present disclosure.

### DETAILED DESCRIPTION

This disclosure is not limited to the particular methodology, materials, and modifications described, and as such, these may vary. Moreover, the terminology used herein is for the purpose of describing particular aspects only and is not intended to limit the scope of the claims. Nor are the claims limited to the disclosed aspects.

Unless defined otherwise, all technical and scientific terms used herein have the same meaning as commonly understood to one of ordinary skill in the art to which this disclosure pertains. Any methods, devices, or materials similar or equivalent to those described herein can be used in the practice or testing of the example embodiments.

As used herein, the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. The term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

The use of “or” in the present application is with respect to a “non-exclusive” arrangement, unless stated otherwise. For example, when stating that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, “and/or” is intended to mean a grammatical conjunction used to indicate that one or more of the elements or conditions recited may be included or occur. For example, a device comprising a first element, a second element and/or a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element.

As used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element. A similar interpretation is intended when the phrase “used in at least one of:” is used herein.

Embodiments of the present disclosure include a cell culture vessel having a sparger that is operable to eliminate the use of three-dimensional (3D) air supply channels to supply air or gas to the bubble emitter holes of a sparger. Embodiments of the present disclosure provide a two-dimensional (2D) film that advantageously reduces the volume occupied by a sparger within a vessel or more particularly a cell culture vessel. Embodiments of the present disclosure are operable to reduce the amount of liquid and cell retention that can enter a sparger's air manifolding under the bubble emitter holes. Further, embodiments of the present disclosure allow for lower cost manufacturing than traditional spargers. Exemplary embodiments reduce the number of fabrication steps in producing a sparger and reduce the amount of material required for manufacturing and, for maintaining flexibility of the sparger. Embodiments therefore reduce the likelihood of fracturing and damage during shipping and use in flexible vessels like bags.

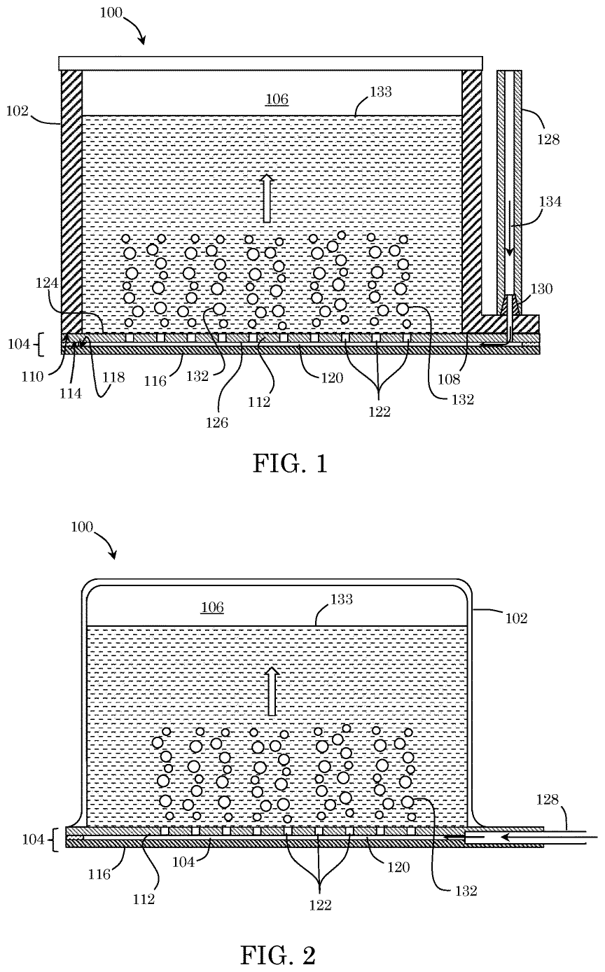

Referring to FIGS. 1 and 2, shown are cross-sectional views of an exemplary cell culture vessel 100 suitable for practicing embodiments of this disclosure. The cell culture vessel 100 comprises a housing 102 and a sparger 104. The housing 102 is operable to maintain a cell culture and fluid. The housing 102 defines a cavity 106 and has an open end 108 fluidly connected to the cavity 106. Embodiments of the housing 102 can be made of a solid material or a flexible material. In some embodiments housing 102 can be made of a transparent material or an opaque material. In some embodiments housing 102 can be made of a glass material or a flexible plastic or polymer material such that housing 102 may be operable to expand in response to air or gas that is moved into the housing 102 and contract in response to air or gas that is moved out of the housing 102. The open end 108 of the housing 102 is surrounded by the peripheral edge 110 of the housing 102. Embodiments of peripheral edge 110 include the peripheral edge 110 being coextensive throughout its entirety such that a planar object will be able to be in contact with the entire extent of the peripheral edge 110 simultaneously.

The sparger 104 may be coupled to the open end 108 at the peripheral edge 110 of the housing 102. The sparger 104 may be sealed to the housing 102 by an air-tight seal between the film 112 and the housing 102 that prevents fluid (e.g., gas, air) from passing between the housing 102 and the sparger 104. The sparger 104 includes a film 112 having a first peripheral edge portion 114 and a plate 116 having a second peripheral edge portion 118. The first peripheral edge portion 114 is the exterior edge of the film 112 facing toward plate 116 and away from housing 102 and peripheral edge 110. The second peripheral edge portion 118 is the exterior edge of plate 116 that faces toward film 112. The first peripheral edge portion 114 of film 112 may be sealed to the second peripheral edge portion 118 such that the remaining portions of the film 112 and the plate 116 are not sealed to one another. In other words, portions of the surfaces of film 112 and plate 116 that are facing one another and are not sealed to each other are spaced apart from one another forming a fluid passageway 120 between the film 112 and the plate 116. Embodiments of film 112 and the plate 116 include the film 112 and the plate 116 being sealed to each other by at least one of an airtight adhesive, heat sealing, heat staking, ultrasonic welding, or solvent bonding. The seal between the film 112 and the plate 116 may be a leak tight seal that prevent fluids, including gases, from passing between the film 112 and the plate 116 along the portions that are sealed together.

Embodiments of film 112 may have a thickness of (i) less than about 2.5 millimeters, (ii) between about 0.005 to about 0.025 inches, (iii) between about 0.025 to about 0.05 inches, or (iv) between about 0.05 to about 0.1 inches. In embodiments, film 112 may not substantially extend into the cavity 106 beyond the peripheral edge 110 of the housing 102 even in the instance that gas or air is moving through the fluid passageway 120 between film 112 and plate 116. Substantially extending into the cavity 106 would include the film 112 crossing the threshold of cavity 106 defined by the plane created by the peripheral edge 110 of the housing 102 by more than the thickness of the film 112. In embodiments, the film 112 may not extend into the cavity 106 beyond (i) more than about 1 micrometer, (ii) more than about 5 micrometers, (iii) more than about 10 micrometers, (iv) more than about 25 micrometers, (v) more than about 50 micrometers, or (vi) more than about 100 micrometers. In embodiments, the film 112 may be spaced apart from the plate 116 by less than about 100 micrometers. In other embodiments, the film 112 may be spaced apart from the plate 116 by (i) less than about 5 micrometers, (ii) between about 5 to about 10 micrometers, (iii) between about 10 to about 25 micrometers, (iv) between about 25 to about 50 micrometers, or (vi) between about 50 to about 100 micrometers.

Embodiments of the film 112 can be made of a polymer. Other exemplary embodiments of film 112 can be made of polystyrene (PS), polycarbonate (PC), polyethylene terephthalate (PET), polypropylene (PP), polyvinylchloride (PVC), or polyetheretherketone (PEEK). Embodiments of the film 112 can be rigid, flexible or semi-flexible based on the characteristics of the film 112 such as thickness, composition and processing during manufacturing.

The film 112 includes a plurality of spaced apart holes 122 that extend through the thickness of the film 112. Each one of the plurality of holes 122 fluidly connects the cavity 106 and the fluid passageway 120 and is operable to allow a gas (e.g., air) to pass therethrough. Embodiments of the plurality of holes 122 include each hole having a diameter of at least one of (i) between about 50 micrometers to about 150 micrometers, (ii) between about 150 micrometers to about 250 micrometers, (iii) between about 250 micrometers to about 500 micrometers, (iv) between about 500 micrometers to about 1 millimeter, or (v) less than about 1 millimeter. Embodiments of the plurality of holes 122 include the holes having a diameter of less than about 100 micrometers and greater than about 100 micrometers.

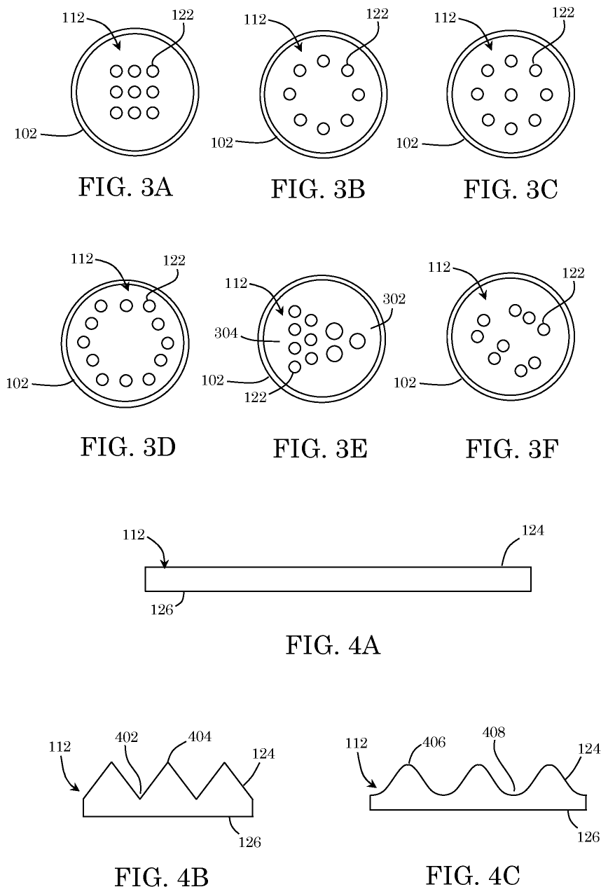

The plurality of holes 122 can be positioned in film 112 in a number of different shapes and arrangements. Referring to FIGS. 3A-3F, shown are some non-limiting embodiments of exemplary arrangements of the plurality of holes 122 illustrated in a top view of the film 112. In embodiments, the plurality of holes 122 may be arranged in a square or rectangular pattern (shown in FIG. 3A). In embodiments, the plurality of holes 122 may be arranged in a ring pattern (shown in FIG. 3B). In embodiments, the plurality of holes 122 may be arranged in a circular or oval pattern (shown in FIG. 3C). In embodiments, the plurality of holes 122 may be arranged in a hexagonal pattern (shown in FIG. 3D). In embodiments, the plurality of holes 122 may be arranged in a triangular pattern (shown in FIG. 3E). In embodiments, the plurality of holes 122 may be arranged in spaced apart groups or islands having two or more holes associated with each grouping (shown in FIG. 3F). It should be appreciated that embodiments include any type of arrangement of the plurality of holes 122 on film 112, including the use of holes 122 with differing diameters within the same film 112 and the use of different spacing between holes 122. In other words, embodiments include the plurality of holes 122 having a different density of holes along film 112 such that holes 122 on a first portion of film 112 may be spaced apart at a first distance from each other and holes 122 on a second portion of film 112 may be spaced apart at a second distance or varying distances that are less than or more than the first distance. For example, in FIG. 3E, embodiments include holes 122 in portion 302 of film 112 having a larger diameter and being spaced farther apart from one another than in the portion 304 of film 112 where the holes 122 have a smaller diameter and are closer together.

Embodiments of film 112 include the surface 124 of film 112 that faces the cavity 106 being treated with a treatment or a material coating prior to being assembled into the cell culture vessel 100. Embodiments of exemplary material coatings or treatments to surface 124 may be operable to inhibit or prevent cell attachment to the surface 124 of film 112. In embodiments, surface 124 may be treated by charging the surface 124 such that it has a net negative surface charge. In embodiments, the surface 124 may be treated such that it has a net positive surface charge. In embodiments, surface 124 of film 112 may be treated with a corona-gas treatment. Embodiments of film 112 include at least one of surface 124 or the surface 126 that faces the plate 116 being treated with a material coating. In embodiments, cell culture vessel 100 may include a film 112 in which surface 124 is selectively treated with a tissue culture treatment or CellBIND surface treatment operable to increase the likelihood for the attachment of cells.

Embodiments of cell culture vessel 100 further include a gas line 128 fluidly connected with fluid passageway 120. Embodiments of gas line 128 allow a gas to pass through gas line 128 into fluid passageway 120 and through the plurality of holes 122. Embodiments may include multiple gas lines 128 being fluidly connected to fluid passageway 120. In this regard, embodiments may include gas (e.g., oxygen) or air from multiple different gas lines 128 being provided to fluid passageway 120 and cavity 106. Embodiments may further include a one-way valve 130 located on the gas line 128 operable to allow a flow of fluid into the fluid passageway 120 in a first direction 134 (i.e., the direction flowing from the gas line 128 into the fluid passageway 120) and prevent a flow of fluid in a second direction (i.e., from the fluid passageway to the gas line 128). In embodiments, the gas line 128 may be fixedly attached between the film 112 and the plate 116 such that the gas line 128 can provide gas to the fluid passageway 120. Gas line 128 may be operable to be removably affixed to a gas source that maintains the gas or air under pressure and is operable to selectively release the gas or air from the gas source.

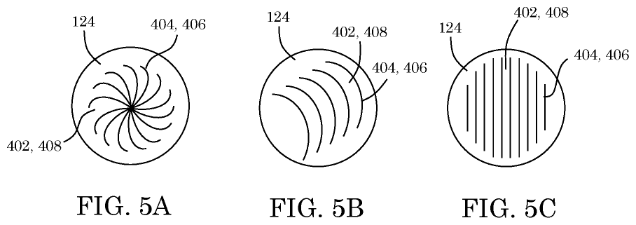

Embodiments of film 112 provide that surface 124 of the film 112 may be planar. In embodiments, surface 124 of film 112 may be non-planar. Referring to FIG. 4A-4C, shown are cross-sectional views of exemplary embodiments of film 112. In the embodiments illustrated in FIGS. 4A-4C, film 112 includes surface 124 that has protrusions 404 while the opposite surface (i.e., the surface of film 112 that faces plate 116) does not have protrusions 404, 406. However, embodiments include both surfaces 124, 126, of film 12 having a plurality of protrusions 404, 406. As shown in FIG. 4A, surface 124 and/or surface 126 may generally be planar and flat. As shown in FIG. 4B, surface 124 may include a plurality of channels 402 separated from each other by a plurality of protrusions 404. In the embodiment shown in FIG. 4B, the channels 402 and the protrusions 404 may have pointed edges at the top of the protrusions 404 and at the bottom of the channels 402. As shown in FIG. 4C, surface 124 may include a plurality of protrusions 406 and channels 408. In the embodiment shown in FIG. 4C, the protrusions 406 and channels 408 may include curved edges at their respective high points and low points.

Embodiments of film 112 include the protrusions 404, 406 that can form or define any type of shape or geometry along surface 124 of film 112 in order to accommodate the purpose desired in the cell culture vessel 100. For example, it may be desired to stir the fluid in the cavity 106 in a counterclockwise fashion about the center of film 112. In this example, the channels 402, 408 and corresponding protrusions 404, 406 can be arranged in a spiral fashion, as shown in FIG. 5A. FIGS. 5B and 5C illustrate non-limiting exemplary arrangements of channels 402, 408 and protrusions 404, 406 on surface 124 of film 112. FIG. 5B illustrates a plurality of protrusions 404, 406 in a crescent shape. FIG. 5C illustrates a plurality of protrusions 404, 406 that appear as lines that are parallel to one another. Embodiments of channels 402, 408 and protrusions 404, 406 can be arranged in any shape or design to accommodate the desired purpose of the user. In the embodiments illustrated in FIGS. 5A-5C, the protrusions 404, 406 are depicted by the lines and the channels 402, 408 are represented by the spaces between the lines.

In practice, prior to assembling the cell culture vessel 100, the surface 124 of film 112 can be treated by a surface treatment and a plurality of holes 122 in film 112 can be formed by, for example, laser ablation. Embodiments include the surface treatment occurring before or after the plurality of holes 122 are formed in film 112. The surface treatment can advantageously be applied to surface 124 after the plurality of holes 122 are formed in film 112 such that the surface treatment can permeate the film 112 along the edges of the plurality of holes 122. The cell culture vessel 100 can then be formed by positioning a film 112 opposite the plate 116 and sealing the first peripheral edge portion 114 of the film 112 to the second peripheral edge portion 118 of the plate 116 such that the film 112 is spaced part from the plate 116 by a distance less than about 100 micrometers to form the fluid passageway 120 between the film 112 and the plate 116. The housing 102 can then be affixed or sealed to film 112. Embodiments of the present disclosure provide that the cell culture vessel 100 is operable to receive a deposit of a cell culture and to maintain a fluid and/or cells within cavity 106. The cells within cavity 106 may require a certain amount of oxygen or other gases (including air) to develop as desired by the user. The cell culture vessel 100 may be operable to provide the desired oxygen or other gases (including air) to cavity 106. Pressured gas can be passed through gas line 128, one-way valve 130, fluid passageway 120 (between film 112 and plate 116), and out the plurality of holes 122 into cavity 106. As gas passes through fluid passageway 120, film 112 does not expand or flex in a manner that causes film 112 to substantially expand into the cavity 106. Rather, the gas that passes through fluid passageway 120 is restricted to pass through fluid passageway 120 between film 112 and plate 116. In this regard the film 112 does not take up any substantial volume within cavity 106. Rather, all, or nearly all of the volume of cavity 106 can be used or occupied by the contents of the housing 102. The pressured gas that enters the cavity 106 is emitted from the plurality of holes 122 in the form of fine gas bubbles 132 that rise through the liquid medium 133 contained in housing 102 (e.g., cavity 106). Although the film 112 does not have a traditional 3D pipe or channel designed to receive gas from gas line 128, the gas is able to flow in the fluid passageway 120 between the film 112 and the plate 116 due to imperfections in the surfaces of the film 112 and the plate 116 that provide a sufficiently low resistance to allow gas to flow due to the very low viscosity of the gas. In other words, embodiments of film 112 and plate 116 do not require the film 112 to deform or expand in response to the pressure from the gas during use to create the fluid passageway 120 that allows the gas to flow.

Embodiments of film 112 have a surface roughness sufficient to allow for gas or air flow between plate 116 and file 112. In embodiments, the surface roughness of film 112 may be due to treating the surface of film 112 with anti-block particles that project or extend from the surface of film 112, thereby creating a rough surface that is not uniformly smooth. Roughness to the surface of film 112 can be imparted by, for example, rollers on a film extrusion line during manufacturing, or from polymer retraction when forming the holes 122 via a perforation method. Embodiments provide that the plurality of holes 122 can be formed by mechanical hole punching or other methods, such as by laser ablation. Conversely, embodiments may include plate 116 having a surface roughness obtained by treating the surface of plate 116 during manufacturing such that the surface of plate 116 does not have a uniformly smooth surface but instead has a plurality of asperities that decrease the surface-to-surface contact with film 112. The roughness of one or both of the surfaces of film 112 and plate 116 provide space between the film 112 and plate 116 such that gas from gas line 128 can pass through fluid passageway 120 between film 112 and plate 116 and through holes 122. Embodiments include having one or multiple holes 122 based on the desired gas flow rate into cavity 106. Embodiments include having an increased number of holes 122 to accommodate higher gas or air flow rates into cavity 106 and a lower number of holes 122 to accommodate lower gas or air flow rates as desired by the user.

Embodiments of spargers 104 can be integrated into many types of vessels used for the culture of cells, such as bioprocess cell expansion bags, spinner flasks, oxygenator vessels, bioreactors and the like.

This disclosure has been described in detail with particular reference to an embodiment, but it will be understood that variations and modifications can be undertaken within the spirit and scope of the disclosure. The presently disclosed embodiments are therefore considered in all respects to be illustrative and not restrictive. The scope of the disclosure is indicated by the appended claims, and all changes that come within the meaning and range of equivalents thereof are intended to be embraced therein

1. A cell culture vessel, comprising:
a housing defining a cavity operable to maintain a cell culture and an open end fluidly connected to the cavity, the open end surrounded by a peripheral edge of the housing;
a sparger coupled to the open end at the peripheral edge, the sparger comprising a film comprising a first peripheral edge portion and a plate comprising a second peripheral edge portion, the first peripheral edge portion sealed to the second peripheral edge portion such that the film is spaced apart from the plate by less than about 100 micrometers to form a fluid passageway between the film and the plate, the film further comprising a plurality of spaced apart holes extending through a thickness of the film, the fluid passageway in fluid communication with the cavity through the plurality of holes.

2. (canceled)

3. The cell culture vessel of claim 1, wherein the film comprises a polymer.

4. The cell culture vessel of claim 1, wherein a diameter of each hole of the plurality of holes is less than about 1 millimeter.

5. The cell culture vessel of claim 1, further comprising a gas line in fluid communication with the fluid passageway.

6. The cell culture vessel of claim 5, further comprising a one-way valve operable to allow a flow of fluid into the fluid passageway in a first direction and prevent a flow of fluid from the fluid passageway in a second direction.

7. (canceled)

8. The cell culture vessel of claim 5, wherein the gas line is fixedly attached between the film and the plate.

9. The cell culture vessel of claim 1, wherein the film comprises a first major surface facing the cavity, and the first major surface is non-planar.

10. The cell culture vessel of claim 9, wherein the first major surface is a treated surface configured to at least one of enhance or inhibit cell attachment to the first major surface.

11. The cell culture vessel of claim 10, wherein the treated first major surface comprises a net negative surface charge.

12. The cell culture vessel of claim 10, wherein the treated first major surface comprises a coating.

13. The cell culture vessel of claim 1, wherein a wall of the housing is flexible.

14. (canceled)

15. A method of sparging a cell culture, comprising
depositing the cell culture into a cell culture vessel, the cell culture vessel comprising:
a housing defining a cavity operable to maintain the cell culture and an open end fluidly connected to the cavity, the open end surrounded by a peripheral edge of the housing;
a sparger coupled to the open end at the peripheral edge, the sparger comprising a film comprising a first peripheral edge portion and a plate comprising a second peripheral edge portion sealed to the first peripheral edge portion such that the film is spaced apart from the plate by less than about 100 μm to form a fluid passageway between the film and the plate, the film further comprising a plurality of spaced apart holes extending through a thickness of the film, the fluid passageway in fluid communication with the cavity through the plurality of holes; and
flowing a gas into the fluid passageway, the gas issuing from the plurality of holes into the housing.

16. The method of claim 15, wherein a diameter of each hole of the plurality of holes is less than about 1 millimeter.

17. The method of claim 15, wherein the gas flows through a gas line extending between the fluid passageway and a gas source.

18.-21. (canceled)

22. A method of forming a cell culture sparger, comprising:
positioning a film comprising a first peripheral edge portion and a plurality of holes extending through a thickness of the film opposite a plate comprising a second peripheral edge portion;
sealing the first peripheral edge portion to the second peripheral edge portion such that the film is spaced apart from the plate by a distance less than about 100 micrometers to form a fluid passageway between the film and the plate.

23. The method of claim 22, wherein the sealing comprises at least one of an airtight adhesive, heat sealing, heat staking, ultrasonic welding, or solvent bonding.

24. The method of claim 22, wherein the thickness of the film is less than about 2.5 millimeters.

25. The method of claim 22, further comprising treating a surface of the film to at least one of enhance or inhibit cell attachment to the surface.

26.-29. (canceled)

30. The method of claim 22, wherein the film comprises a first major surface facing the fluid passageway and a second major surface opposite the first major surface, the second major surface comprising one or more protrusions extending therefrom.

31. The method of claim 22, wherein the film comprises a first major surface facing the fluid passageway and a second major surface opposite the first major surface, and the second major surface is non-planar.
